(19) United States
(12) Patent Application Publication
(10) Pub. No.: US 2009/0112144 A1
(43) Pub. Date: Apr. 30, 2009

(54) Open Mesh Crochet Device

(75) Inventor: Carol Boyer, Shoreview, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Carol Boyer, Shoreview, MN (US)

(21) Appl. No.: 11/924,672

(22) Filed: Oct. 26, 2007

(51) Int. Cl. A61F 13/00

(52) U.S. Cl. 602/60

(57) ABSTRACT

In at least one embodiment, the invention is directed to a method of improving the flow of lymphatic fluid. In one embodiment, the flow of lymphatic fluid is improved by softening fibrotic or scar tissue. The method of improving the flow of lymphatic fluid comprises placing a first layer of open mesh composition/material on the affected area and then placing a second layer over the first layer, where the second layer compresses the first layer so that the first layer forms indentations in, and raised areas or hills on the affected area, thereby massaging the affected area to enhance flow of lymphatic fluid. In one embodiment, massaging the affected area loosens fibrotic tissue, thereby enhancing the flow of lymphatic fluid.

### CROSS-REFERENCE TO RELATED APPLICATIONS

Not Applicable

### STATEMENT REGARDING FEDERALLY SPONSORED RESEARCH

Not Applicable

### FIELD OF THE INVENTION

The invention relates to a method of improving the flow of lymphatic fluid. One means to improve the flow of lymphatic fluid is to soften fibrotic or scar tissue.

### BACKGROUND OF THE INVENTION

The lymphatic system is part of the circulatory system and consists of lymphatic vessels, mph nodes and other lymphoid tissues. The lymphatic system is a drainage system that cleans and removes waste and excess fluid from the tissues. Fluid, or lymph, is transported by a system of lymphatic vessels located under the skin to facilitate flow of fluid to a deeper network of vessels, then to lymph nodes, where the lymph is filtered and cleaned. Thereafter the cleaned and filtered lymph is returned to the blood stream.

The lymph is pumped in and out of vessels by muscle movement and by contraction of the walls of larger vessels. In some cases the lymphatic system malfunctions and causes lymphedema, an abnormal accumulation of high protein fluid in the tissues. One out of every forty people in the world may be affected by lymphedema, a progressive chronic condition. Complications of lymphedema include fibrosis or scar tissue formation, infections, cellulits, sepsis, gangrene, cancer of the soft tissue, increased pain due to the compression of nerves caused by fibrosis and increased fluid, loss of function due to swelling, depression due to the disfigurement caused by increased limb girth, deep vein thrombosis due to the pressure of the swelling and fibrosis, skin complications, and chronic inflammation.

To reduce the risk of complications, especially fibrosis and the risk of infection, the amount of accumulated protein in the tissues needs to be reduced. One way to reduce the amount of accumulated protein at locations affected by fibrosis, is to establish new drainage pathways for the lymphatic system. New drainage pathways can be made when the fibrotic tissue is softened, for example, by complete decongestive therapy. Complete decongestive therapy includes gradient compression bandaging, manual lymph drainage, skin care to prevent infection and exacerbation of the fibrosis, wound care and exercises to stimulate the lymphatic system.

One current treatment for lymphedema is to soften fibrosis with a device containing foam or other type of padding. The foam/padding moves slightly as the patient moves, thereby massaging the body tissue. Massaging the fibrotic area enhances the lymphatic circulation and facilitates alignment of collagen fibers to decrease fibrosis. These devices are designed to treat either small areas of the body or large areas of the body such as an arm or a leg. These devices have several disadvantages. The devices are bulky and are difficult or impossible to be worn under clothing and can significantly limit function. In addition, the devices are hot and therefore uncomfortable to wear in warm temperatures. Moreover, when these devices are washed, the foam/padding may take several days to dry, thereby requiring a patient to purchase multiple devices for continuous treatment. These disadvantages reduce full compliance with recommended treatment protocols. Furthermore, since patients with lymphedema do not have normal lymphatic flow pattern, the devices may not direct the lymphatic flow to an area with a desired location or type of drainage.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, the invention is directed to a method of improving the flow of lymphatic fluid. In one embodiment, the flow of lymphatic fluid is improved by softening fibrotic or scar tissue. The method of improving the flow of lymphatic fluid comprises placing a first layer of open mesh composition/material on the affected area and then placing a second layer over the first layer, where the second layer compresses the first layer so that the first layer forms indentations in, and raised areas or hills on the affected area, thereby massaging the affected area to enhance flow of lymphatic fluid. In one embodiment, massaging the affected area loosens fibrotic tissue, thereby enhancing the flow of lymphatic fluid.

These and other embodiments which characterize the invention are pointed out with particularity in the claims annexed hereto and forming a part hereof. However, for further understanding of the invention, its advantages and objectives obtained by its use, reference can be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a flat view of a first open mesh design.

FIG. 2 is a flat view of a second open mesh design.

FIG. 3A-B are back and front views of a tubular open mesh layer configured to fit on a leg.

FIG. 4 is a side view of a tubular open mesh layer configured to fit on an arm.

FIG. 5A-B are top views of a tubular open mesh layer configured to fit on a hand.

FIG. 6A-D are top views of flat open mesh layers having different shapes and different open mesh designs.

FIG. 7 is a top view of a second layer.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

The invention is directed towards a method of improving lymphatic flow. In some embodiments, the invention is directed towards a method of softening fibrosis that can occur, for example due to lymphedema, and thereby improve the flow of lymphatic fluid. All stages of fibrosis formation from early to acute to chronic can be softened using this inventive method. In at least one embodiment, the inventive method utilizes lymph vessels/pathways present in the affected tissue, softens and/or eliminates fibrotic tissue, reduces swelling of affected body tissue, prevents the reaccumulation of lymph fluid, prevents and/or eliminates infections, and/or maintain normal/near normal size of a limb or body part. In some embodiments, the inventive method is used in conjunction with other aspects of complete decongestive therapy, e.g. manual lymph drainage, skin care to prevent infection and exacerbation of the fibrosis, wound care and exercises to stimulate the lymphatic system, to reduce swelling of affected body tissue, prevent the reaccumulation of lymph fluid, prevent and/or eliminate infections, and/or maintain normal/near normal size of a limb or body part.

To improve the flow of lymphatic fluid, a first layer is placed on an affected area of the body, e.g. an area of the body that is swollen or has fibrotic tissue. The first layer is an open mesh layer 10. Then a second layer 30 is placed on top of the open mesh layer 10 so that the second layer 30 compresses the first layer, i.e. the open mesh layer 10. Compression of the open mesh layer 10 causes indentations, or valleys, and hills to form in the affected body tissue. By forming the indentations, or valleys, and hills in the affected body tissue when under compression, the open mesh layer 10 massages the affected body tissue, which improves the flow of lymphatic fluid. In at least one embodiment, massaging the affected body tissue softens fibrosis/scar tissue, thereby improving the flow of lymphatic fluid. Note that the depth of the indentations affects ability of the open mesh layer 10 to massage the body tissue when compressed. Thus, the formation of deeper indentations softens fibrosis better than the formation of shallow indentations. The depth of the indentations is dependent on the size of the diameter or width of the strands 12 of the open mesh layer 10, the level of compression executed by the second layer 30 and the length of time the second layer 30 compresses the open mesh layer 10. The open mesh layer 10 can be compressed for up to twenty-four (24) hours at a time.

Thus, the second layer 30 can be considered to be a compression layer. The second layer 30 can be bandaging, nonelastic extremity binders, compression stocking or any other device used to compress the open mesh layer 10. FIG. 7 shows a second layer 30 that is in the form of a bandage 32. Types of bandaging that can be used include, but are not limited to, gauze bandage, short stretch compression bandage, long stretch compression band, triangular bandage, and tube bandage. Examples of non-elastic extremity binders include, but are not limited to, Circ-aid® and the ReidSleeve®. To facilitate pulling on the compression stocking over the open mesh layer 10, the open mesh layer 10 can be engaged by sewing or any other means to the inside of the compression stocking so that the open mesh layer 10/compression stocking can be pulled on at the same time instead of one after the other. The compression layer can provide even pressure or can provide pressure in a gradient, i.e. gradient pressure, at certain locations as desired.

The open mesh layer 10 can have any design so long as when the open mesh layer 10 is under compression, indentations/valleys and hills are formed in the body tissue. Some non-limiting examples of designs that can be used for the open mesh layer 10 are shown in the figures. Any type of stitching, weaving, or other forms of sewing can be used to make the open mesh layer 10. In one embodiment, the open mesh layer 10 is made using a garter stitch.

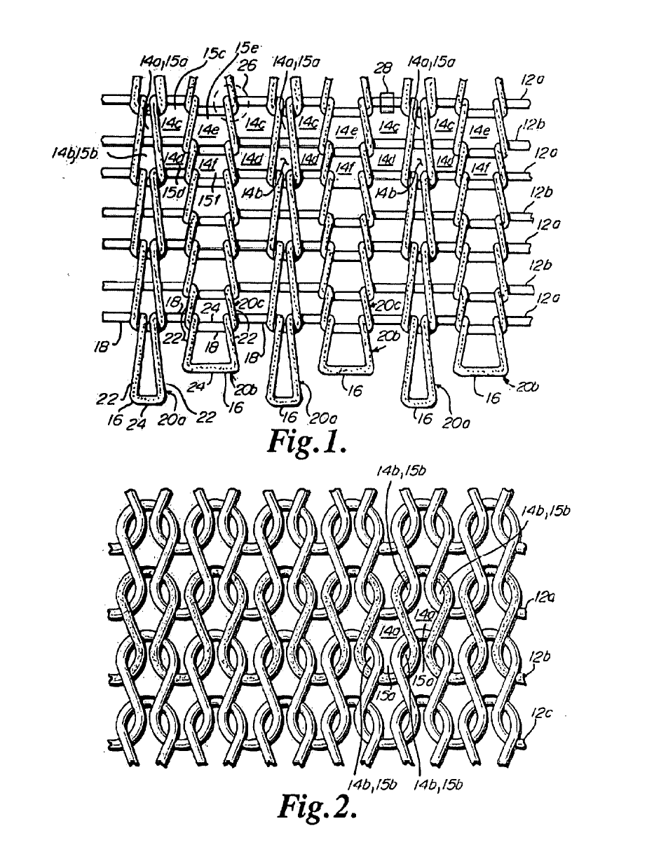

As shown in the figures the open mesh layer 10 comprises a plurality of strands 12 defining a plurality of openings 14. In FIG. 1, the interwoven strands 12 forming the open mesh layer 10 have two different configurations. One group of strands 12a form a first plurality of loops 20a,b and a second group of strands 12b form a second plurality of loops 20c where the size of the first plurality of loops 20a,b is greater than the size of the second plurality of loops 20c. Each of the loops 20a,b,c has two sides 22 and an end 24. Note that the configuration of loops 20b is similar to the configuration of loops 20c with the lengths of the sides 22 being different in loops 20b and loops 20c. The configuration of loops 20a and 20b/c differ in the length of the end 24 of the loop 20 and in the length of the sides 22 of loop 20. In contrast, the interwoven strands 12 forming the open mesh layer of FIG. 2 each have the same configuration. In this embodiment, the strands 12 are in a serpentine configuration, as shown by stippled strand 12b.

As shown in the figures, the openings 14 defined by the strands 12 can have any shape, for example, but not limited to, square-shaped, rectangular shaped, triangular shaped, circular shaped, oval shaped, egg-shaped, eye shaped, irregular shaped and any combination thereof. In at least one embodiment, the spacing of the design of the open mesh layer 10 ranges from about 0.5 cm by 0.5 cm to about 1.0 cm by 1.0 cm. Thus, the size of the openings 14 of the open mesh layer 10 are about 0.5 cm by 0.5 cm to about 1.0 cm by 1.0 cm in size. In some embodiments, the openings 14 have a diameter of about 0.5 cm to about 1.5 cm. In other embodiments, the openings 14 have a length ranging from about 0.5 cm to about 1.5 cm and a width ranging from about 0.5 cm to about 1.5 cm.

In some embodiments, the spacing of design, i.e. the size of the openings 14, is chosen so that the two layer system is cool when worn underneath clothing. In other embodiments, the spacing of the open mesh design is chosen so that the two layer system is not bulky, thereby mitigating the effect the two layer system has on the mobility of the region of the body being treated.

Note that the size of the opening 14 can be considered as the area of the opening 14. The openings 14 of an open mesh layer 10 can all be the same size/area or different sizes/areas. Thus, there can be one, two, three, four, five, six, seven, eight, nine, ten or more sizes/areas of the openings 14 of the open mesh layer 10. Note that the size/area of the openings 14 determines the size/area of the hills formed on the affected body tissue when the open mesh layer 10 is compressed. In addition, the size/area of the openings 14 determines the number of indentations formed when the open mesh layer 10 is compressed. Thus, an open mesh layer 10 with larger openings 14 makes fewer indentations when compressed than an open mesh layer 10 with smaller openings 14. In other words, the greater the percentage of area of openings 14 to the total area of the open mesh layer 20, the fewer indentations formed.

The open mesh layer 10 can have openings 14 with a plurality of different configurations 15 and/or sizes/areas, as shown for example in FIG. 1. Note that openings can have different configurations 15 but the same area. FIG. 1 has six different openings 14a,b,c,d,e,f having different configurations 15a,b,c,d,e,f and/or sizes. As can be seen in FIG. 2, the configuration 15e of opening 14e is similar to the configuration 15f of opening 14f, but the size of opening 14e is greater than the size of opening 14f. In this open mesh design, each opening 14 is defined by a plurality of strands 12. For example, opening 14a is defined by three different strands 12, and opening 14c is defined by four different strands 12. Thus, it is within the scope of the invention for an opening 14 in an open mesh layer 10 to be defined by one, two, three, four, five, six, seven, eight, nine, ten or more strands 12.

In FIG. 2, the open mesh layer 10 has openings 14 with two different configurations 15 and/or sizes. For example, the open mesh layer 10 has a first opening 14a with a first configuration 15a and a second opening 14b with a second configuration 15b, where the first configuration 15a is different than the second configuration 15b and the first opening 14a is greater in size than the second opening 14b. In this open mesh design, each of the first openings 14a are defined by three different strands 12 and each of the second openings 14b are defined by two different strands 12.

In at least one embodiment, first portions 16 of the open mesh layer 10 are raised compared to second portions 18 of the open mesh layer 10. This is shown, for example, in FIG. 1 where the first portions 16 of the open mesh layer 10 that are stippled are raised compared to the second portions 18 of the open mesh layer 10 that are not stippled, i.e. the non-raised portions. In this embodiment, when the open mesh layer 10 is compressed with the raised first portions 16 against the body tissue, the raised first portions 16 form deeper indentations than the nonraised second portions 18 of the open mesh layer 10.

In other embodiments, areas of the open mesh layer 10 that have more material, e.g. due to the overlap of strands 12 of material, form deeper indentations than areas of the open mesh layer 10 that only have one strand 12 of material. This is due to the fact that areas with overlapping strands 12 are thicker than areas without overlapping strands 12 and the thicker portions are pressed deeper into the body tissue when compressed. The circled area 26 of FIG. 1 is a non-limiting example an area of the open mesh layer 10 where at least two strands 12 of material overlap and therefore form deeper indentations that the rectangular bound area 28 of the open mesh layer 10. Thus, under compression, an open mesh layer 10 can make indentations of varying depth depending upon the number of strands 12 of material that overlap and the level of compression applied to the open mesh layer 10.

In some embodiments, the open mesh layer 10 has only one design. In other embodiments, the open mesh layer 10 has more than one design. In this embodiment, for example, a portion of the open mesh layer 10 has a first design, e.g. the design shown in FIG. 1, and a portion of the open mesh layer 10 has a second design, e.g. the design shown in FIG. 2. It is within the scope of the invention for the open mesh layer 10 to have one, two, three, four, five, six or more open mesh designs. The designs of the open mesh layer 10 can be tailored to the severity of the fibrosis, i.e. fibrosis that extends deeper into the tissue is more severe than fibrosis that does not extend as deep. Thus, an open mesh design that causes deep indentations under compression can be used for areas with deep fibrosis, and an open mesh design that causes shallower indentations under compression can be used for areas with shallow fibrosis.

In at least one embodiment, the open mesh layer 10 is reversible, i.e. the pattern of indentations formed when one side of the open mesh layer 10 is compressed against the body tissue is different than the pattern of indentations formed when the opposite side of the open mesh layer 10 is compressed against the body tissue. This can occur when the design on one side of the open mesh layer 10 is different from the design on the opposite side of the open mesh layer 10. For example, the indentations made by the open mesh layer 10 of FIG. 1 will produce one pattern of indentations when the open mesh layer 10 is placed with the stippled raised first portions 16 against the body tissue and another pattern of indentations when the open mesh layer 10 is placed with the stippled raised first portions 16 away from the body tissue.

A reversible open mesh layer 10 can be made by making an open mesh layer 10 with two layers, each having a different open mesh design, instead of one layer. In this embodiment, one layer forms one side of the open mesh layer 10 and another layer forms the opposite side of the open mesh layer 10. The two layers are attached to one another. Thus, when the orientation of the reversible open mesh layer 10 is alternated over time, a reversible open mesh layer 10 can soften more body tissue in a given area than a nonreversible open mesh layer 10 because different patterns of indentations will be formed in the same area. For example, where hills were formed when one design is compressed, indentations are formed when the second design is compressed and vice-versa. Note that for some one layer open mesh layer 10 designs the pattern of indentations is the same but the depth of the indentations formed can vary depending on which side of the open mesh layer 10 is against the body tissue and/or the amount of compression used. Thus, a one layer open mesh layer 10 can also soften more body tissue in a given area when the orientation of the one mesh open layer 10 is alternated. The same effect can also be achieved by alternating the use of open mesh layers 10 with different open mesh designs.

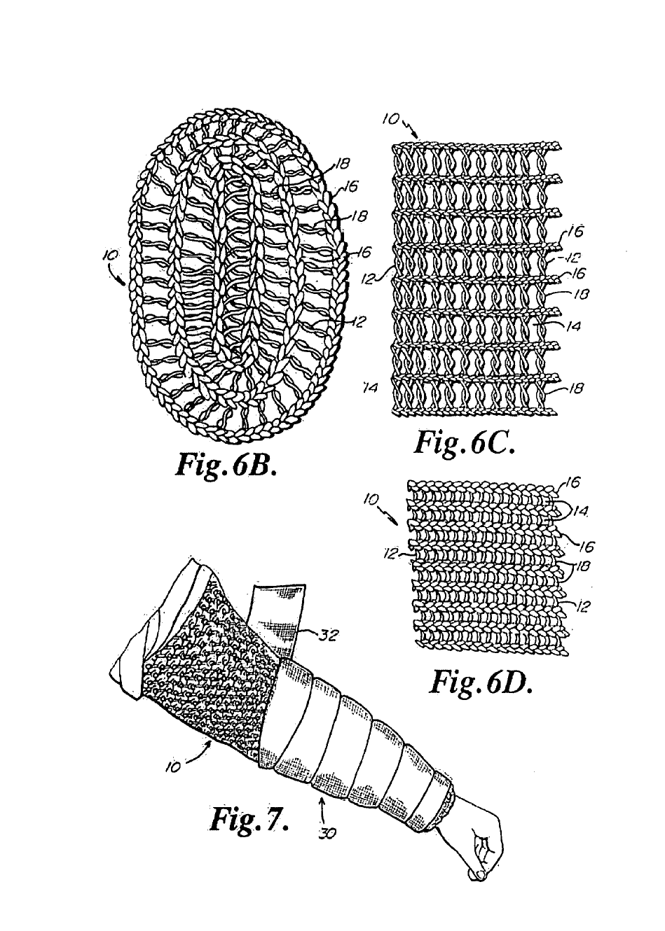

In at least one embodiment, the open mesh layer 10 is flat, e.g. like a scarf or bandage, and can have any size and shape, for example, round, oval, square, rectangular, and triangular, as shown in FIG. 6A-D. If the open mesh layer 10 is in the form of a scarf or bandage, i.e. long and narrow, the open mesh layer 10 can be wrapped about a portion of the body in a manner similar to a bandage that is wrapped around a portion of the body. Alternatively, the open mesh layer 10 can be a portion of a tubular open mesh layer 10, which is described in greater detail below. Thus, in this embodiment, the open mesh layer 10 can be one-quarter, one-third, one-half, two-thirds, or any other fraction of a tubular open mesh layer 10. For example, if only the front portion of a leg 50 requires therapy, an open mesh layer 10 that is half the size of a tubular open mesh layer 10 for a leg 50, can be placed on the front portion of the leg 50 and compressed. In some embodiments, the open mesh layer 10 is about 4 inches to about 6 inches wide.

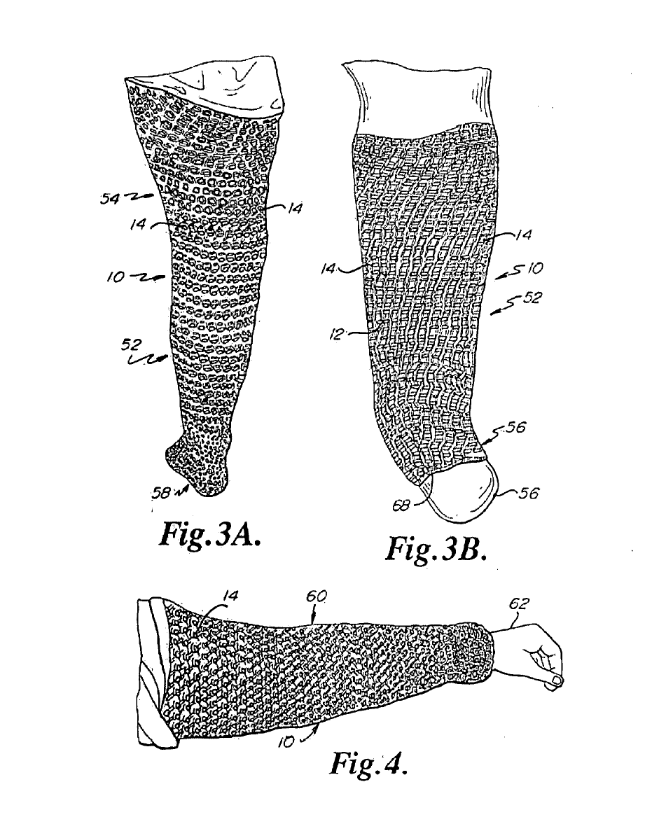

In at least one embodiment, the open mesh layer 10 is tubular. A tubular open mesh layer 10 can be worn on a leg 50 or arm 56, as shown for example in FIGS. 3A-B and 4. The tubular open mesh layer 10 can have any circumference so that it can fit over different sizes of legs 50 and arms 56. Note that because the open mesh layer 10 is compressed, the tubular open mesh layer 10 does not require a snug fit to form indentations. Thus, as the swelling of the body tissue decreases, a new smaller tubular open mesh layer 10 is not required. The tubular open mesh layer 10 can have any length so that it can be worn over all or a portion of the arm 56 or leg 50.

The tubular open mesh layer 10 in FIG. 3A-B, shown being worn on a leg 50, extends from about the middle of the thigh 54 of the leg to the middle of the foot 56. However, the tubular open mesh layer 10 could extend only down to the ankle. The tubular open mesh layer 10 in FIG. 4 that is shown being worn on an arm 60 extends from the upper arm near the shoulder, i.e. the armpit region, down to the wrist. In some embodiments, the tubular open mesh layer 10 is reinforced in areas that receive more wear and tear, e.g. heel region 58 of the foot 56 or the elbow region of the arm 60, thereby increasing the useful life of the open mesh layer 10.

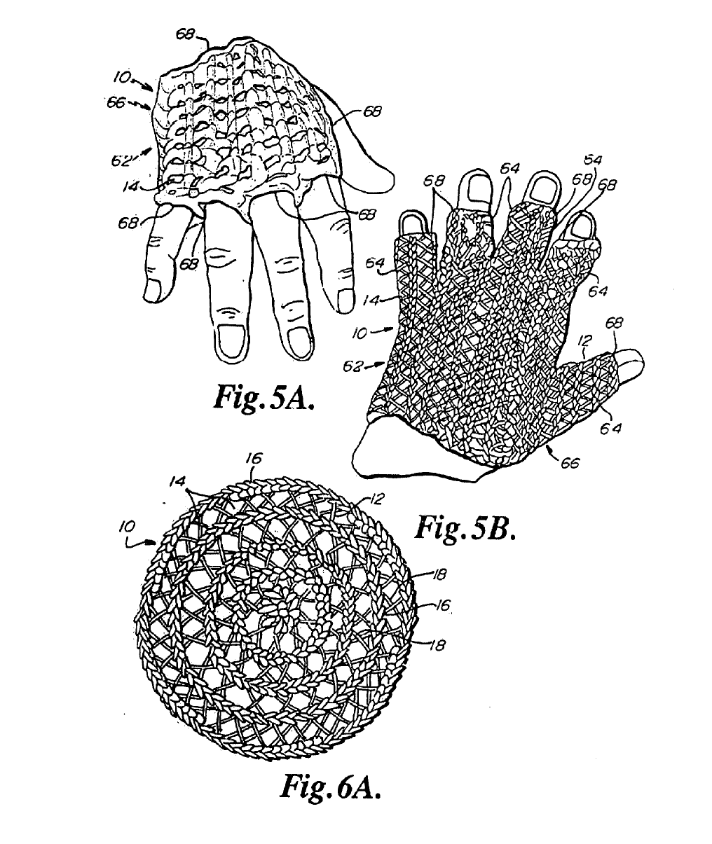

In at least one embodiment, the open mesh layer 10 is tubular with at least one opening 68 for fingers 64 or toes so that the open mesh layer 10 can be worn on a foot 56, as shown in FIGS. 3A and B, or on a hand 62, like a fingerless glove 66, as shown for example in FIG. 5A. Thus, the tubular open mesh layer 10 can have one opening 68 as shown in FIG. 3A, openings 68 for each finger/toe 64, as shown in FIG. 5A, or there can be fewer than five openings 68 so that any chafing around the finger/toe 64 can be minimized yet the position of the glove 66 or sock on the hand 62 or foot 56 can be maintained. It is within the scope of the invention for the tubular open mesh layer 10 shown in FIG. 4 to be paired with the glove 66 of FIG. 5A-B. In some embodiments, the tubular mesh layer 10 can be made into a glove 66 with at least one finger 64, shown in FIG. 5B. Although the fingers 64 in FIG. 5B are open at the ends, it is within the scope of the invention for the fingers 64 to be closed at the ends. Note that although the glove 66 in FIG. 5B has five fingers 64, it is within the scope of the invention for the glove to have one, two, three, four, five, or more fingers 64. Thus, if only a portion of the fingers 64 is affected by lymphedema, only those affected finger 64 can be encompassed by a finger 64 of the glove 66.

Although cotton is preferred because it is less likely to cause allergic reactions, the strands 12 forming the open mesh layer 10 can be made from any type of material, for example, but not limited to, cotton, wool, silk, nylon, polyester, spandex, latex, acrylic, and any combination thereof. The strands 12 forming the open mesh layer 10 can be made from the same material or different materials. In at least one embodiment, the strands 12 forming the open mesh layer 10 are yarn. In one embodiment, the yarn is made of cotton. It is within the scope of the invention for the yarn to be any ply, e.g. two, four, six, eight ply. In one embodiment the open mesh layer 10 is made from 4 ply yarn. The yarn can have any diameter ranging from about 0.1 cm to about 0.3 cm. In at least one embodiment, the yarn has a diameter greater than 0.2 cm. Note that the diameter will affect the size of the indentations formed by the open mesh layer 10. In some embodiments, the material used to make the open mesh layer 10 has a neutral color to prevent allergic reactions to dye. In other embodiments, the open mesh layer 10 is made from a material that can wick moisture away from the body. In at least one embodiment, the open mesh layer 10 is made from a material that can be machine washed and machine dried for convenience.

The open mesh layer 10 can be made by any means, for example, but not limited to, knitting machines, crocheting machines, knitting by hand, crocheting by hand and any combination thereof. In some embodiments, the open mesh layer 10 is hand crocheted using a crochet hook sized J, K or N. The open mesh layer 10 can be made using either a single crochet method or a double crochet method.

In at least one embodiment, a tubular mesh layer 10 for the leg 50 is made by first crocheting a chain equal to the circumference of the foot 56 at the base of the toes 57 and then joining the chain to form a loop. Then you crochet around the chain with a single crochet to form a base the first round. The second and subsequent rounds are crocheted through loops (both top) for a nubbier effect with a single or double crochet pattern increasing as needed to encompass the leg 50 as needed up to the knee, thigh 54 or other desired location on the leg 50. Thus, the tubular open mesh layer 10 for a leg 50 can have a length ranging from about eight (8) inches to about sixty (60) inches and a circumference ranging from about six (6) inches to about forty (40) inches. The tubular mesh layer 10 for the arm 60 can be made in a similar manner except that the initial chain has a length equal to the circumference of the wrist. The tubular mesh layer 10 can be crocheted up to the elbow or up to the armpit. Thus, the tubular open mesh layer 10 for an arm 60 can have a length ranging from about four (4) inches to about thirty (30) inches and a circumference ranging from about four (4) inches to about thirty (30) inches.

In at least one embodiment, the tubular mesh layer 10 with openings 68 for the fingers 64 is made by crocheting a rectangle that begins with a chain that has a length equal to the distance from the base of the fingers to the wrist, i.e. the width of the rectangle. The width of the rectangle can range from about two (2) inches to about ten (10) inches. The rectangle is crocheted until it has a length equal to the distance from the thumb across the palm and around the hand back to the thumb. The length of the rectangle can range from about four (4) inches to about fifteen (15) inches. When the rectangle has the appropriate length, the two ends are crocheted together leaving an opening 68 for the thumb. One edge of the rectangle is crocheted together so that there are openings 68 for fingers 64. If a glove 66 with fingers 64 is desired, open or closed tubes can be crocheted by starting at the opening and crocheting until the desired length is reached. If an open mesh layer 10 for the arm 60 and hand 62 is desired, the other edge of the rectangle can be crocheted onto a tubular open mesh layer 10 made for the arm 60 as described above.

A circular flat open mesh layer 10 can be made by crocheting a chain of two stitches, crochet five single or double crochet stitches for form a first loop. Then a first round is formed by crocheting two stitches in each stitch through both top loops for a total often stitches. In subsequent rounds, every other stitch is increased in order to maintain a flat circular piece. The crocheting continues around in a circle, with the increasing to keep the open mesh layer 10 flat, until the circular flat open mesh layer 10 has the desired diameter. It is within the scope of the invention for the diameter of the circular flat open mesh layer to be about two (2) inches to about twenty (20) inches. In one embodiment the diameter of the circular flat open mesh layer is about four (4) to about six (6) inches.

An oval shaped flat open mesh layer 10 can be made by crocheting a chain with an appropriate length to cover a fibrotic area, e.g. one half of the fibrotic area in order to account for increasing at each end of the device, and crocheting around each side of the chain in single or double crochet in both top loops increasing every other stitch in curves at each end as needed to maintain a flat oval shape. First round is crocheted in single crochet for a base and increase three stitches at each end for an oval shape. Subsequent rounds are crocheted in single or double crochet increasing as needed on each end. It is within the scope of the invention for the oval shaped flat open mesh layer 10 to be about two (2) inches to about fifty (50) inches in length and about two (2) inches to about fifty (50) inches wide.

A square or rectangular shaped flat open mesh layer 10 can be made by first crocheting a chain having a length equal to the width of the fibrotic area. Crochet in both top loops, in single or double crochet, turning at the end of each row and measuring a length to form a square or rectangle to cover a fibrotic area. It is within the scope of the invention for the square or rectangular shaped flat open mesh layer 10 to be about two (2) inches to about fifty (50) inches in length and about two (2) inches to about fifty (50) inches wide.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A method of softening fibrotic tissue comprising:
acquiring a first layer, the first layer being designed to cover at least a portion of the fibrotic tissue, the first layer having a length of about two (2) inches to about sixty inches (60) and a width of about two (2) inches to about sixty inches (60), the first layer having at least one open mesh design, the open mesh design comprising a plurality of strands, the plurality of strands defining a plurality of openings, each strand having a diameter of about 0.1 cm to about 0.3 cm, each opening having a length of about 0.5 cm to about 1.5 cm and a width of about 0.5 cm to about 1.5 cm, each strand being made from a material selected from at least one member of the group consisting of natural fibers, synthetic fibers, cotton, wool, silk, nylon, polyester, spandex, latex, acrylic, and any combination thereof;
acquiring a second layer, the second layer being selected from at least one of compression stocking, bandage, non-elastic compression device and any combination thereof;
covering at least a portion of the fibrotic tissue with at least a portion of the first layer;
compressing at least a portion of the first layer with at least a portion of the second layer into the at least a portion of the fibrotic tissue thereby forming hills and valleys in the at least a portion of the fibrotic tissue.

2. A method of improving the flow of lymphatic fluid in an affected area comprising:
placing a first layer on at least a portion of the affected area, the first layer having at least one open mesh design; and
compressing at least a portion of the first layer with a second layer placed at least partially over the first layer so that the at least one open mesh design of the first layer forms indentations and hills in the affected area.

3. The method of claim 2, the first layer being at least one of flat and tubular.

4. The method of claim 2, the second layer being selected from at least one of compression stocking, bandage, nonelastic compression device and any combination thereof.

5. The method of claim 4, the bandage being selected from the group consisting of gauze bandage, short stretch compression bandage, long stretch compression bandage, triangular bandage, tube bandage, and any combination thereof.

6. The method of claim 2, the open mesh design comprising a plurality of strands, the plurality of strands defining a plurality of openings, each opening having a length of about 0.5 cm to about 1.5 cm and a width of about 0.5 cm to about 1.5 cm.

7. The method of claim 6, each opening having a length of about 0.5 cm to about 1 cm and a width of about 0.5 cm to about 1 cm.

8. The method of claim 6, the at least one open mesh design being made by a plurality of strands, each strand having a diameter of about 0.1 cm to about 0.3 cm

9. The method of claim 8, each of the plurality of strands being made from a material selected from at least one member of the group consisting of cotton, wool, silk, nylon, polyester, spandex, latex, acrylic, and any combination thereof.

10. The method of claim 8, the first layer being made of 4 ply cotton yarn having a diameter of at least 0.2 cm.

11. The method of claim 8, wherein compressing the first layer with a second layer results in a gradient of pressure being applied to the first layer so that the pressure gradually increases along the first layer.

12. The method of claim 8, the tubular first layer being configured to be placed on at least a portion of an arm, a leg or a hand.

13. The method of claim 12, the tubular first layer configured to be placed on at least a portion of a leg having a length from about eight (8) inches to about sixty (60) inches and a circumference ranging from about six (6) inches to about forty (40) inches.

14. The method of claim 12, the tubular first layer configured to be placed on at least a portion of an arm having a length ranging from about four (4) inches to about thirty (30) inches and a circumference ranging from about four (4) inches to about thirty (30) inches.

15. The method of claim 12, the tubular first layer configured to be placed on at least a portion of a hand having at least one opening for fingers to extend therethrough.

16. The method of claim 12, tubular first layer configured to be placed on at least a portion of a hand being in the form of a glove.

17. The method of claim 16, the glove having tubular portions extending about at least one finger of the hand.

18. The method of claim 2, the flat first layer having a shape selected from at least one member of the group consisting of round-shaped, oval-shaped, square-shaped, rectangular shaped, triangular shaped and any combination thereof.

19. The method of claim 18, the flat first layer having a width of about two (2) inches to about sixty (60) inches and a length of about two (2) inches to about sixty (60) inches.

20. The method of claim 18, the flat first layer having a width of about 4 inches to about 6 inches and a length of about four (4) inches to about six (6) inches.
